(19) United States
(12) Patent Application Publication
(10) Pub. No.: US 2006/0045891 A1
(43) Pub. Date: Mar. 2, 2006

(54) Density-matched suspension vehicles and pharmaceutical suspensions

(76) Inventor: Phillip Michael Lovalenti, Palo Alto, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/199,933

(22) Filed: Aug. 9, 2005

Related U.S. Application Data
(60) Provisional application No. 60/604,307, filed on Aug. 24, 2004.

(51) Int. Cl. A61K 9/00

(52) U.S. Cl. 424/400

(57) ABSTRACT

Density-matching is used to provide suspending vehicles, pharmaceutical suspensions, dosage forms, and kits as well as methods of making and using the vehicles, suspensions, and dosage forms. Pharmaceutical suspensions comprising a pharmaceutically active agent having an active agent density, ρ_{A}, and a suspending vehicle having a suspending vehicle density, ρ_{SV}; wherein the suspending vehicle density, ρ_{SV} is substantially equal to the active agent density, ρ_{A}, are provided. Suspending vehicles comprise at least one suspending agent. The suspending vehicles can further comprise at least one density-modifying solid in such a combination with the suspending agent as to create a suspending vehicle that has a density that substantially matches the density of a desired drug particle or combination of drug particles. Pharmaceutical suspensions that remain homogenous during prolonged storage can be obtained.

### CROSS-REFERENCE TO RELATED APPLICATIONS

This claims the benefit of priority under 35 U.S.C. § 119(e) from provisional U.S. Application Ser. No. 60/604,307, filed on Aug. 24, 2004, which is incorporated herein by reference in its entirety.

### FIELD OF THE INVENTION

The present invention relates to suspending vehicles and pharmaceutical suspensions in drug delivery systems and drug dosage forms utilizing the same, and more particularly to pharmaceutical suspensions that remain homogenous during prolonged storage.

### BACKGROUND OF THE INVENTION

Ensuring stability of pharmaceutical agents within dosage forms that include suspensions is important, for example, for effective dosaging and/or shelf-stability. Pharmaceutical suspensions can be used, for example, in osmotic, also referred to as pump-driven, drug delivery devices and injection depot devices.

One approach to providing a stable suspension of a pharmaceutical agent has been to provide a dosage form containing a suspending vehicle having a viscosity that is sufficiently high to slow the sedimentation rate of the pharmaceutical agent, whose density is typically higher than that of the suspending vehicle. Settling of particles in a suspension can be characterized by Stokes' equation:

$$V = ( \frac{2 g {R}^{2}}{9 μ} ) ( {ρ}_{P} - {ρ}_{C} ) \qquad ( A )$$

wherein V is velocity of settling, μ is the viscosity of the carrier, g is acceleration due to gravity, ρₚ is the density of the particle, ρ_{c} is the density of the carrier, and R is the particle radius of the pharmaceutically active agent. U.S. Pat. No. 5,972,370, incorporated herein by reference, discusses that the viscosity of pharmaceutical suspensions can be altered by the use of thickening agents to raise the suspension's viscosity to the desired level. U.S. Patent Application Pub. No. 2003/0191192 discusses the use of suspending polymers in aqueous pharmaceutical suspensions for oral administration.

Suspensions that are very high in viscosity (for example, 10,000 to 20,000 poise), however, put a significant burden on, for example, processing equipment used to load the delivery devices with such vehicles. In applications utilizing non-aqueous, parenterally-acceptable suspension formulations or vehicles, this typically limits the choices of vehicles to polymer solutions, such as polyvinyl pyrolidone (PVP) in a solvent, which tends to result in phase separation and to pluggage of, for example, discharge ports of implantable devices after exposure to bodily fluids.

Another approach to enhancing the stability of suspension dosage forms is to minimize the average particle size of the dispersed phase, for example, the pharmaceutically active agent. According to the Seventh Edition of “Pharmaceutical Dosage Forms and Drug Delivery Systems” (Ansel et al. at 350), “the physical stability of a pharmaceutical suspension appears to be most appropriately adjusted by an alteration in the dispersed phase rather than through great changes in the dispersion medium.” From a manufacturing standpoint, however, increased losses and difficulty in handling occur as the desired size of drug particles decreases. In addition, for certain types of drugs such as proteins and peptides, the types of processes that can produce very small drug particle sizes, while maintaining the integrity of the drug, are limited.

Colloidal suspensions of drug particles can be used in suspension dosage forms, but the use of submicron drug particle sizes is required, resulting in the aforementioned manufacturing issues of losses and difficulty in handling. Further, not all types of drugs can be prepared on a submicron level. For example, proteins and peptides are typically not prepared as submicron particles, because the processing involved cannot ensure that their physical and therapeutic integrities will be preserved.

Hence, there exists a need for ways to use a wider range of suspending agents in extended-release suspension formulations while maintaining physical stability and effective dosaging over the long term. There also exists a need for ways to utilize relatively large pharmaceutical agent particle sizes that remain suspended within suspension dosage forms. Additionally, there is a need for suspending vehicles that are resistant to phase separation, and for dosage forms which remain substantially homogenous for months and even years at a time.

### SUMMARY OF THE INVENTION

In one aspect, the present invention provides methods for preparing pharmaceutical suspensions by substantially matching a density of a desired active agent with a density of a suspending vehicle. The present invention also provides suspending vehicles, dosage forms, and kits, as well as methods of making and using the vehicles, suspensions, and dosage forms.

Pharmaceutical suspensions according to the invention generally comprise at least one pharmaceutically active agent having an active agent density, ρ_{A}, and at least one suspending vehicle having a suspending vehicle density, ρ_{SV}. In preferred embodiments, the suspending vehicle density, ρ_{SV}, is substantially equal to the active agent density, ρ_{A}.

Suspending vehicles according to the invention generally comprise at least one suspending agent. The suspending vehicles can further comprise at least one density-modifying solid in such a combination with the suspending agent as to create a suspending vehicle that has a bulk density that substantially matches the density of a desired drug particle. A desired drug particle may comprise a single active pharmaceutical or a combination of drugs and/or inactive ingredients, with the idea being that the pharmaceutically active drug particles to be suspended are of a substantially uniform density from particle to particle. Differences in densities between the suspending vehicle and the drug particle can be 0.1 g/cc or less, preferably 0.05 g/cc or less, more preferably 0.01 g/cc or less. Also, it is preferable for certain applications, such as implantable devices for delivering proteins and/or peptides, that the suspending vehicle or suspending agent be substantially non-aqueous.

Through density-matching, pharmaceutical suspensions that remain homogenous during prolonged storage can be obtained. Preferred suspensions according to the present invention remain substantially homogenous at room temperature without shaking for at least one month, preferably up to six months, and even more preferably up to one year or more.

The pharmaceutical suspensions of the present invention can be incorporated into a wide variety of pharmaceutical dosage forms that contain a suspension. For example, osmotic/pump-driven devices generally would benefit from the pharmaceutical suspensions of the present invention, as would injection depot-type devices.

In certain embodiments of the invention, a dosage form for delivering a pharmaceutically active agent to a biological environment of use comprises: a first wall that maintains its physical and chemical integrity during the life of the dosage form and is substantially impermeable to a pharmaceutical suspension; a second wall that is partially permeable to an exterior fluid present in the biological environment; a compartment defined by the first wall and the second wall; a pharmaceutical suspension that is positioned within the compartment and comprises a pharmaceutically active agent having an active agent density, ρ_{A}, and a suspending vehicle having a suspending vehicle density, ρ_{SV}; wherein the suspending vehicle density, ρ_{SV}, is substantially equal to the active agent density, ρ_{A}; a pump in communication with the first wall, the second wall, and the compartment; and an exit port in the wall in communication with the compartment. Preferably, the dosage form comprises an osmotic pump.

Kits in accordance with the present invention comprise containers holding a suspending vehicle and instructions for mixing the suspending vehicle with a pharmaceutically active agent. In addition, other kits comprise a suspending agent and a density-modifying solid with instructions on how to mix them to match the density of the active agent particles to be suspended.

Methods provided by the present invention generally comprise: identifying at least one pharmaceutically active agent having an active agent density, ρ_{A}; identifying a suspending vehicle having a suspending vehicle density, ρ_{SV}; and determining whether the active agent density, ρ_{A}, differs from the suspending vehicle density, ρ_{SV}. At least one pharmaceutically active agent can be mixed with the suspending vehicle to create a pharmaceutical suspension.

In other embodiments, methods further comprise: identifying at least one suspending agent having a suspending agent density, ρ_{SA}; and identifying at least one density-modifying solid having a solid density, ρ_{P}; and mixing the at least one suspending agent and the at least one density-modifying solid to create the suspending vehicle. Methods can also include calculating a difference between the active agent density, ρ_{A}, and the suspending vehicle density, ρ_{SV}. If the difference is substantial, then an additional amount of the suspending agent or density-modifying solid can be added to the suspending vehicle. Adjustments may also be made during the preparation of suspending vehicles or pharmaceutical suspensions by adding suspending agents or density-modifying solids other than the ones used at the outset.

The methods can further comprise determining a weight fraction of the density-modifying solid, X_{P}, in the suspending vehicle, wherein the suspending vehicle density, ρ_{SV}, and the active agent density, ρ_{A} are substantially the same.

An additional optional step in the production of suspending vehicles can include centrifugation to settle out those particles that are subject to excessive sedimentation, retaining the supernatant as a “stabilized” suspending medium. This step can also aid in removing entrapped air bubbles created during mixing. Preparation of submicron particles of density-modifying solids can involve grinding the particles in the presence of the intended suspending liquid, such as in a stirred media mill. In this way, a particle generation and mixing step would be combined.

Other methods of, for example, preparing pharmaceutical suspensions using suspending vehicles of known densities, comprise: identifying a suspending vehicle having a known density, ρ_{SV}, and mixing at least one pharmaceutically active agent having an active agent density, ρ_{A}, with the suspending vehicle to create a pharmaceutical suspension; wherein the known density, ρ_{SV}, is substantially the same as the active agent density, ρ_{A}.

Further methods of, for example, preparing suspending vehicles intended for density-matching, comprise identifying at least one suspending agent having a suspending agent density, ρ_{SA}; identifying at least one density-modifying solid having a solid density, ρ_{P}; mixing the suspending agent with the density-modifying solid to create a suspending vehicle having a vehicle density, ρ_{SV}; and establishing a weight fraction of the density-modifying solid, X_{P}, in the suspending vehicle, such that the vehicle density, ρ_{SV}, is from about 1.0 g/cc to about 2.5 g/cc. The methods can further comprise determining whether the suspending vehicle density, ρ_{SV}, differs from an active agent density, ρ_{A}, of a pharmaceutically active agent; and mixing the pharmaceutically active agent with the suspending vehicle to create a pharmaceutical suspension. It may be desirable to identify a biologic-compatible suspending agent, for use in conjunction with proteins and peptides.

Still further methods comprise administering pharmaceutical suspensions and/or dosage forms containing the pharmaceutical suspensions to a mammal.

### BRIEF DESCRIPTION OF THE DRAWINGS

FIG. 1 depicts a container holding a suspending vehicle in accordance with the present invention.

FIG. 2 depicts a container holding a pharmaceutical suspension in accordance with the present invention.

FIG. 3 illustrates an osmotic pump-driven dosage form holding a pharmaceutical suspension in accordance with the present invention.

### DETAILED DESCRIPTION OF ILLUSTRATIVE EMBODIMENTS

In accordance with one aspect of the invention, the density of a suspending vehicle, _{SV}, is substantially matched to the density of a pharmaceutically active agent, _{A}. Densities of suspending vehicles can be tailored to the density of a desired active agent. As such, preferred pharmaceutical suspensions comprise a pharmaceutically active agent having an active agent density, ρ_{A}, and a suspending vehicle having a suspending vehicle density, ρ_{SV}, wherein the suspending vehicle density, ρ_{SV} is substantially equal to the active agent density, ρ_{A}.

Suspending vehicles comprise at least one suspending agent. The suspending vehicles preferably further comprise at least one density-modifying solid in such a combination with the suspending agent as to create a suspending vehicle that has a density that substantially matches the density of a desired drug particle or combination of drug particles. In preferred embodiments, the suspending vehicles are combined with drug particles to create pharmaceutical suspensions that are parenterally acceptable.

By reference to densities that are substantially equal, substantially matched, substantially the same, or insubstantially different, it is understood that there may be some difference between the densities of a suspending vehicle and the drug particles, but the differences can be 0.1 g/cc or less, preferably 0.05 g/cc or less, even more preferably 0.01 g/cc or less. A substantial difference in densities would lead to inhomogeneity in the suspension after a certain period of time, for example, one month.

Reference to suspending agent and suspending vehicle means that the pharmaceutically active agent and density-modifying solid are substantially insoluble therein. Materials that are substantially insoluble generally remain in their original physical form throughout the lifespan of a dosage form containing the suspension. For example, solid particulates would generally remain particles. If necessary, the suspending agent or suspending vehicle may have other materials dissolved in them, and their respective densities, ρ_{SA}, and ρ_{SV} would reflect any addition materials.

Reference to “density-modifying solid” means a solid that is substantially inert in the suspending agent or suspending vehicle. As such, a density-modifying solid has a minimal effect on the stability or effectiveness of a desired pharmaceutically active agent.

The term “pharmaceutically active agent” includes but is not limited to pharmaceuticals and drug particles which require delivery to a mammal to achieve a therapeutic response. This term also includes drugs that are accompanied by excipients or processing aids in their solid particulate form.

As to suspensions that are parenterally-acceptable, it is understood that there would be no adverse biological responses by the recipient of the suspension upon parenteral administration.

If a desired active agent has been identified, then a suspending vehicle generally can be made so that its density _{SV} substantially matches the active agent density. When at least one density-modifying solid particle is used in conjunction with a suspending agent, a bulk density of a suspending vehicle, ρ_{SV}, can be calculated according to formula (1):

$${ρ}_{SV} = {( \frac{{X}_{P}}{{ρ}_{P}} + \frac{( 1 - {X}_{P} )}{{ρ}_{SA}} )}^{- 1} , \qquad ( 1 )$$

where _{P} is the density of the solid particle; _{SA} is the density of the suspending agent; and Xₚ is the weight fraction of the density-modifying solid particle in the suspending vehicle. As such, based on the densities of the solid particle and suspending agent and their respective amounts, a bulk density of the resulting suspending vehicle density can be predicted.

If more than one suspending agent is utilized, then ρ_{SA} would be the density of a mixture of suspending agents. If necessary, the suspending agent or suspending vehicle may have other materials dissolved in them, and their respective densities, ρ_{SA} and ρ_{SV}, would reflect any additional materials.

If more than one density-modifying solid is utilized, then ρ_{P} would be the volume-averaged density of a mixture of the solids. Rearranging formula (1), the weight fraction of the density-modifying solid, Xₚ, in the suspending vehicle can determined according to formula (2):

$${X}_{P} = \frac{{ρ}_{P} * ( {ρ}_{SA} - {ρ}_{SV} )}{{ρ}_{SV} * ( {ρ}_{SA} - {ρ}_{P} )} , \qquad ( 2 ) .$$

Based on substantially matching the suspending vehicle density, _{SV}, to a density of a pharmaceutically active agent, _{A}, and knowing the densities of the solid, _{P}, and the suspending agent, _{SA}, a weight fraction of the solid in the suspension can be predicted. Should more than one pharmaceutically active agent be used or should there be other desirable excipients or inactive agents associated with the pharmaceutically active agent, then the density of the pharmaceutically active agent, _{A}, would be the density of a particle that is a combination of all of the desired ingredients.

It is also possible to prepare a variety of suspending vehicles comprising a suspending agent and a density-modifying solid oÿer a range of known densities, for example, from about 1.0 g/cc to 2.5 g/cc for ultimate use in the preparation of pharmaceutical suspensions. In this type of situation, a suspending vehicle would be chosen for its known density, and also perhaps for its suitability for a particular type of active agent, for example, a small molecular drug, a protein, or a peptide.

Suspending Vehicles

The vehicle formulation strategy of the present invention provides a tunable suspension density to substantially match that of an active agent, for example, a drug particle. In so doing, physically stable suspension drug formulations (stable for months to over a year) can be produced by relieving the constraints on vehicle viscosity and drug particle sizing associated with other approaches. Typically, suspending vehicles comprise ingredients that are compatible for long-term storage and dosaging of active agents.

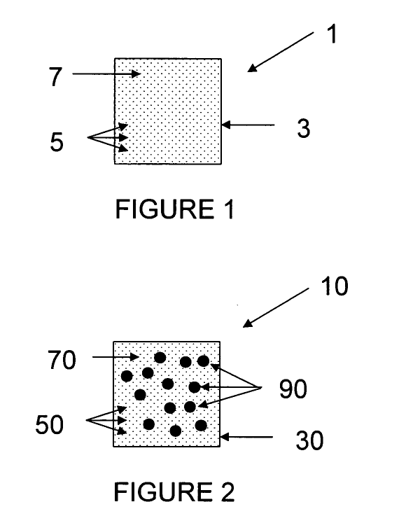

Referring now to FIG. 1, a suspending vehicle 1 according to the present invention comprises a suspending agent 7 and a plurality of nanoparticles of a density-modifying solid 5 in a container 3. A drug manufacturer who desires to utilize a suspending vehicle can provide, for example, a density and optionally a particle size of a drug it desires to deliver and a suspending vehicle such as that represented in FIG. 1 can be prepared according to the present invention for use by the manufacturer. A kit in accordance with the present invention comprises a suspending vehicle which has a density to substantially match that of a desired pharmaceutically active agent. Optionally, instructions for mixing the pharmaceutically active agent and the suspending vehicle are provided. Another kit in accordance with the present invention comprises a suspending agent and a density-modifying agent. In a preferred embodiment, the kit includes instructions for preparing a mixture of the suspending agent and the density-modifying solid to form a suspending vehicle having a density that is substantially equal to an active agent density.

Pharmaceutical Suspensions

Pharmaceutical suspensions can be created by mixing the pharmaceutically active agent with the suspending vehicle. A pharmaceutical suspension of the present invention can comprise from about 5 to about 25% by weight of the pharmaceutically active agent. Although not intending to be limiting, the pharmaceutical suspensions prepared according to density-matching can be less viscous that those suspensions made otherwise. Less viscous pharmaceutical suspensions are generally easier for processing equipment to handle, for example, for degassing purposes or for loading dosage forms.

With reference to FIG. 2, a pharmaceutical suspension 10 comprises a suspending agent 70, a plurality of particles of a density-modifying solid 50, and particles of a pharmaceutically active agent 90 in a container 30. In some embodiments of the present invention, pharmaceutical suspensions can comprise: 40-99.5 wt % suspending vehicle, 0.5-50 wt % active agent, and 0-10 wt % surfactant. The suspending vehicle can comprise 0.75 wt % density-modifying solids and 25-100 wt % suspending agent.

Dense Particles

A general advantage to using dense, substantially inert particles to modify densities of pharmaceutical suspensions is that the burden of smallness and sedimentation rates can be shifted from the pharmaceutically active agent particles (which can have limitations on smallness) to the dense, substantially inert particles. Typically, the dense particles, also referred to as density-modifying particles, are more dense than the pharmaceutically active agent particles. Dense particles can be amenable to a variety of manufacturing processes (such as cryo or mill grinding or precipitation processing) possibly not available to the pharmaceutically active agent particles because of chemical and/or physical stability limitations of the active drug.

By mixing small dense particles in a suspending liquid, a bulk density can be produced which is between the density of the pure liquid (lower limit) and that of a maximum packed suspension (upper limit). A bulk density of this mixture can be matched to the density of a drug particle by changing the amount of dense particles in the mixture. The mixtures as such can be used as vehicles for making stable suspension drug formulations.

The use of dense, substantially inert particles in combination with a fluid to create a suspension formulation or vehicle may in some cases be limited by maximum packing limitations on the weight fraction of particles. As such, a suspending fluid can be chosen with a viscosity that can compensate for a small mismatch between the suspending vehicle and the drug particles, thereby still producing a stable suspension drug formulation and providing a broader array of choices for appropriate suspending liquids compared to having not used dense particles.

It is preferable that density-modifying solids have minimal effect on the stability or effectiveness of a desired pharmaceutically active agent. As such, some solids may be suitable for certain active agents, but not suitable for others. For example, proteins or peptides that are pH sensitive and perhaps denature in an acidic environment would likely not be amenable to preparation with an acidic density-modifying agent.

If more than one density-modifying solid is utilized, then pp of formulas (1) and (2) would represent the volume-averaged density of solids in a mixture.

It is preferable, but not limited as such, that the solid comprise submicron particles. An average particle size of the solid is from about 100 nm to about 700 nm. The density of the solid, ρₚ, is preferably greater than 2.0 g/cc, and more preferably greater than 2.5 g/cc. Exemplary density-modifying solids include, but are not limited to, talc, metal acetates, metal ascorbates, metal carbonates, metal chlorides, metal oxides, metal phosphates, metal silicates, metal stearates, metal sulfates, or combinations thereof. Preferably, the metals include, but are not limited to: calcium, magnesium, potassium, sodium, or zinc. In preferred embodiments, the density-modifying solid comprises a zinc phosphate, a zinc silicate, a calcium phosphate, a calcium silicate, a calcium carbonate, a zinc chloride, a sodium silicate, or combinations thereof. In other embodiments, the density-modifying solid comprises calcium acetate, calcium ascorbate, calcium chloride, calcium sulfate, calcium stearate, magnesium silicate, magnesium stearate, potassium carbonate, potassium chloride, potassium phosphate, sodium acetate, sodium carbonate, sodium chloride, sodium phosphate, sodium sulfate, zinc acetate, zinc carbonate, zinc chloride, zinc oxide, zinc oxide, zinc stearate, zinc sulfate, or combinations thereof.

Average particle sizes of the density-modifying particles are preferably less than about 700 nm, more preferably from about 100 nm to 500 nm, and even more preferably from about 200 nm to about 400 nm.

The density-modifying solids should be of sufficient density greater than desired drug particles so as to raise the density of a suspending medium without taking up excessive volume within the dosage form. It is preferable that the small dense particles are substantially inert to drug particles and the suspending medium to ensure compatibility with a wide range of drug particles and long-term chemical stability. They should be parenterally acceptable and, ultimately, will require FDA approval for parenteral administration. In addition, dense particles that are substantially insoluble in water are desirable to aid in limiting water ingress to a suspension dosage form, which in turn enhances chemical stability and desired dosaging by reducing degradation of drug particles. It is also preferable that the small dense particles are capable of being produced on a submicron level.

Zinc phosphates and silicates have very low water solubility and densities in the range of about 3.0 to about 4.0 g/cc. As such, these types of dense particles would require less volume in a suspension dosage form than other particles of lesser densities to give a suspending vehicle a density boost. In terms of particle size, nanoparticles of zinc phosphates and silicates can be potentially produced by precipitation of the salts out of an aqueous medium. It is possible that such zinc salts could be parenterally acceptable since zinc-protein chelates are already parenterally approved.

Calcium phosphates and silicates have low water solubility and densities in the range of about 2.4 to about 3.3 g/cc. Accordingly, such dense particles would require less volume in a suspension dosage form than other particles of lesser densities in order to give a suspending vehicle a density boost. In terms of particle size, nanoparticles of calcium phosphates and silicates can be potentially produced by precipitation of the salts out of an aqueous medium. It is possible that such calcium salts could be parenterally acceptable due to their measurable water solubility and ability to be eliminated by dissolution.

Zinc chloride, with a density of approximately 2.9 g/cc, is a parenterally approved material. Nanoparticles of zinc chloride could be manufactured through mill grinding or cryogrinding, though it is not likely they can be produced by precipitation of the salts out of an aqueous medium due to its water solubility. Although zinc chloride is water soluble, and as a result, may not help to reduce water ingress into dosage forms such as osmotic devices, it is possible that for certain applications it would be desirable as a density-matching material.

Nanoparticles of dense materials can be produced as understood in the art, see for example U.S. Pat. No. 6,623,761 and U.S. Patent Application Pub. No.: 2003/0003155, both of which are incorporated herein by reference.

Suspending Agents

Suspending agents are capable of suspending a desired pharmaceutically active agent and a density-modifying solid. As such, the active agent and solid are substantially insoluble therein. The suspending agents should have minimal effect on the stability or effectiveness of a desired pharmaceutically active agent. Suspending agents should be acceptable for desired routes of administration, for exampÿe, parenterally-acceptable, and preferably, FDA-approved.

If more than one suspending agent is utilized, then ρ_{SA} would represent the density of a mixture of suspending agents. If necessary, the suspending agent or suspending vehicle may have other materials dissolved in them, and their respective densities, ρ_{SA}, and ρ_{SV} would reflect any additional materials.

Exemplary suspending agents include, but are not limited to, Vitamin E/alpha tocopherol, vegetable oils, lipids, or combinations thereof. Examples of vegetable oils include, but are not limited to, cottonseed oil, peanut oil, sesame oil, and soybean oil. Desirable lipids include, but are not limited to: phosphatidylcholine (PC), phosphatidylethanolamine (PE), phosphatidylglycerol (PG), phosphatidylinositol (PI), phosphatidylserine (PS), phosphatidic acid (PA), sphingomyelin (SM). Some examples of individual lipids include: neutral lipids—Dioleoyl phosphatidylcholine (DOPC), dimyritoylphosphatidylcholine (DMPC), dipalmitoylphosphatidylcholine (DPPC), distearoylphosphatidylcholine (DSPC), egg phosphatidylcholine (egg PC), soy phosphatidylcholine (soy PC), partially or fully hydrogenated phosphatidylcholins (PHSPC or HSPC), palmitoyl-oleoyl phosphatidylcholine (POPC), stearoyl-oleoylphosphatidylcholin (SOPC); and anionic lipids Dioleoy phosphatidylglyserol (DOPG), imyritoylphosphatidylglyerol (DMPG), Dipalmitoylphosphatidylglyserol (DPPG), distearoylphosphatidylglyserol (DSPG). Various PEG (polyethyleneglycol)-lipids can also be included for added benefit. Examples of PEG-lipids are mPEG-DPPE, mPEG-DMPE, mPEG-DSPE, -mPEG-ceramide-DSPE. Also several Pluronics like F-127, Spans, etc. can be used. Increments of anti-oxidant lipid agents such as vitamin E/alpha tocopherol can be added to along with lipids to prevent oxidation or formation of peroxides.

It is desirable that suspending agents have low water solubility, which can reduce water ingress into dosaÿe forms such as osmotic devices. Vitamin E has a viscosity of approximately 2,000 centipoise at room temperature (approximately 700 centipoise at body temperature and approximately 10,000 centipoise at 4° C.) and a density of approximately 0.95 g/cc. Vegetable oils typically have a viscosity of less than 100 centipoise at room temperature and a density of approximately 0.2 g/cc. Density of the suspending agent, ρ_{SA}, is preferably greater than or equal to 0. g/c c. In a situation where an agent has a ρ_{SA} that substantially equals ρ_{A}, then the amount of density-modifying solid would be close to zero.

In a preferred form of the invention, dense (approximately 2.0-4.0 g/cc) submicron (approximately 0.1-0.7 micron) substantially water-insoluble, substantially inert particles can be combined with a substantially water insoluble suspending fluid (such as an oil) having a density of approximately 0.9-1.2 g/cc and a viscosity of approximately 100-10,000 centipoise. In order to hinder agglomeration of the submicron particles a surfactant such as SPAN-40 can optionally be included in the formulation. The dense particles, substantially water insoluble liquid, and optional surfactant components would need to be parenterally-acceptable, and ultimately, FDA-approved.

Utilizing density-matching, in contrast to increasing viscosity, as a way to stabilize drug suspensions, permits a wider selection of suspending agents for creating suspension formulations, for example, lower viscosity suspending agents can be used. Viscosity requirements can differ by orders of magnitude depending on the length of time that a particle needs to be suspended. Likewise, the viscosity can differ by an orders of magnitude depending on the particle size of a drug particle or on the difference in density between the drug particle and suspending vehicle. A benefit of utilizing density-matching, and consequently relatively lower viscosity suspending agents, is easing manufacture of suspension dosage forms. For example, degassing operations on dosage forms containing suspending vehicles are facilitated by low viscosity.

Pharmaceutically Active Agents

In accordance with the present invention, any type of pharmaceutically active agent can be utilized. The pharmaceutically active agent can be suspended in any practical form; for example, it is preferable to make micron-sized particulates of an active agent for suspension. It may also be desirable to utilize microsphere dosage forms of a particular drug where the microspheres can contain other pharmaceutically desired excipients or inactive materials that may aid in, for example, releasing the active drug from a dosage form.

With respect to density, ρ_{A} can be at least that of ρ_{SA}, preferably ρ_{A} is from about 1.0 to 2.5 g/cc, more preferably from 1.0 to 1.7 g/cc. In other embodiments, it is preferable that ρ_{A} is from about 1.7 to about 2.5 g/cc. An average particle size of the pharmaceutically active agent is preferably less than about 10 microns, but it is understood that particle size can increase as the difference between ρ_{A} and ρ_{SV} is minimized. Relatively large particles of pharmaceutically active agents can be used in conjunction with certain embodiments of the present invention with an advantage of reduced manufacturing complications of high losses and handling difficulties typically associated with operating in small-size regimes.

In some embodiments, the pharmaceutically active agent comprises a small drug molecule, whereas in others, it may be preferable that the active agent is a protein and/or a peptide. The pharmaceutically active agent can also be any combination of active materials suitable for the application.

With respect to pharmaceutically active agents, liquid compositions preferably comprise from about 0.5% to about 50% pharmaceutically active agent by weight, more preferably from about 1% to about 40%, and even more preferably from about 5% to about 25%.

In some embodiments, the pharmaceutically active agent included in a suspension according to the present invention is generally degradable in water but generally stable as a dry powder at ambient and physiological temperatures. Active agents that may be incorporated into a suspension according to the invention include, but are not limited to, peptides, proteins, nucleotides, polymers of amino acids or nucleic acid residues, hormones, viruses, antibodies, etc. that are naturally derived, synthetically produced, or recombinantly produced.

The active agents included in a suspension according to the present invention may also include lipoproteins and post translationally modified forms, e.g., glycosylated proteins, as well as proteins or protein substances which have D-amino acids, modified, derivatized or non-naturally occurring amino acids in the D- or L-configuration and/or peptomimetic units as part of their structure. Specific examples of materials that may be included in as the pharmaceutically active agent in a suspension of the present invention include, but are not limited to, baclofen, GDNF, neurotrophic factors, conatonkin G, Ziconotide, clonidine, axokine, anitsense oligonucleotides, adrenocorticotropic hormone, angiotensin I and II, atrial natriuretic peptide, bombesin, bradykinin, calcitonin, cerebellin, dynorphin N, alpha and beta endorphin, endothelin, enkephalin, epidermal growth factor, fertirelin, follicular gonadotropin releasing peptide, galanin, glucagon, gonadorelin, gonadotropin, goserelin, growth hormone releasing peptide, histrelin, insulin, interferons, leuprolide, LHRH, motilin, nafarerlin, neurotensin, oxytocin, relaxin, somatostatin, substance P, tumor necrosis factor, triptorelin, vasopressin, growth hormone, nerve growth factor, blood clotting factors, ribozymes, and antisense oligonucleotides. Analogs, derivatives, antagonists agonists and pharmaceutically acceptable salts of each of the above mentioned active agents may also be used in formulating an active agent suspension of the present invention. Preferably, the active agents provided in a suspension of the present invention exhibits little or no solubility in the chosen suspension vehicle.

The active agents can be in various forms, such as uncharged molecules, molecular complexes, pharmacologically acceptable acid or base addition salts such as hydrochlorides, hydrobromides, sulfate, laurylate, palmitate, phosphate, nitrate, borate, acetate, maleate, tartrate, oleate, and salicylate. For acidic drugs, salts of metals, amines or organic cations, for example quaternary ammonium can be used. Derivatives of drugs such as esters, ethers and amides can be used alone or mixed with other drugs. Also, a drug that is water insoluble can be used in a form that on its release from a device, is converted by enzymes, hydrolyzed by body pH or other metabolic processes to the original form, or to a biologically active form.

With respect to pharmaceutically acceptable excipients and other processing aids, it is preferable that the drug particles incorporate any such excipients and/or aids into the solid drug particulate to be delivered from a suspension dosage form. As such, reference to drug particles or pharmaceutically active agents, includes any such excipients or aids incorporated therein. As such, reference to a density of a drug particle or pharmaceutically active agent reflects the density of a particulate material to be delivered from a suspension dosage form, the solid particles may reflect ingredients in addition to a therapeutic drug, so long as the particles are of substantially uniform density from particle to particle.

Dosage Forms

Suspending vehicles and pharmaceutical suspensions can be prepared using density-matching for all types of dosage forms, e.g., oral suspensions, ophthalmologic suspensions, implant suspensions, injection suspensions, and infusion suspensions. A preferred dosage form is an implantable osmotic dosage form such as those described in U.S. Pat. Nos. 5,985,305; 6,113,938; 6,132,420, 6,156,331; 6,395,292, each of which is incorporated herein by reference.

It is preferable that the suspending vehicle, the suspending agent, and/or the density-modifying solid are physiologically acceptable for a desired route of administration, for example, there are no adverse biological responses by the recipient of the suspension upon administration. It is understood that the suspending vehicles, suspending agents, and/or density-modifying solids would also have to be FDA-approved. In some embodiments of the present invention, it is preferable that the components are suitable for parenteral routes of administration, including but not limited to injection, infusion, or implantation.

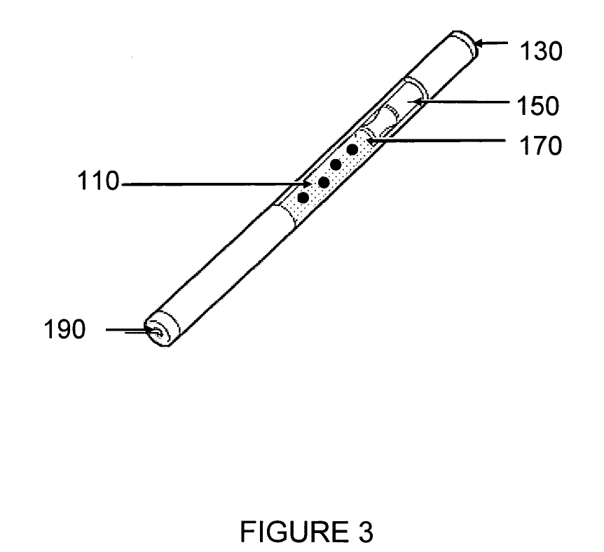

With reference to FIG. 3, it may be desirable for certain dosage forms, for example, an implantable, osmotic dosage form 100, to preload a pharmaceutical suspension according to the present invention comprising a suspending agent, a density-modifying solid, and a pharmaceutically active agent into a drug reservoir 110. Other features of an osmotic dosage form include a semi-permeable membrane 130, an osmotic pump 150, a piston 170, and an exit port 190. In a detailed embodiment of the invention, the dosage form comprises a first wall that maintains its physical and chemical integrity during the life of the dosage form and is substantially impermeable to a pharmaceutical suspension; a second wall that is partially permeable to an exterior fluid; a compartment defined by the first wall and the second wall; a pharmaceutical suspension that is positioned within the compartment and comprises a pharmaceutically active agent and a suspending vehicle whose density is substantially equal to the active agent density; a pump in communication with the first wall, the second wall, and the compartment; and an exit port in communication with the compartment. Preferably, the dosage form comprises an osmotic pump.

Test Methods

The USP does not provide testing protocols for evaluating homogeneity of suspensions over time. Homogeneity over time of suspensions set forth in the following examples were tested using a centrifuge apparatus, referred to herein as the “Centrifuge Test”. The suspensions (about 0.2 mL) are loaded into several small plastic tubes (3-4 cm in length, 3 mm in inner diameter). The tubes are placed in a centrifuge and exposed up to 3000 times gravitational force in the axial direction at temperatures from 4-40° C., for 30 minutes for several hours to accelerate settling, with the intention of simulating about 60 days to several years of settling at normal gravitational forces. The centrifuged tubes of suspensions are sectioned into 2-3 mm sections. The contents of each section are assayed for drug content (or equivalent content of a component of the drug particle). As a result, the concentration profile of the drug along the length of the tube is established. Deviations from the original drug particle concentration represent the degree of sedimentation of the drug particles. Deviations of less than 10% from the original drug concentration following centrifugation of the sample suspension for the required simulated settling time indicate that the suspension remains substantially homogeneous for the time required for the desired application.

### EXAMPLES

Below are several examples of specific embodiments for carrying out the present invention. The examples are offered for illustrative purposes only, and are not intended to limit the scope of the present invention in any way.

### Example 1

### Suspension of Spheres

Precipitated calcium carbonate particles (density 2.71 g/cc and average particle size 0.7 microns) were mixed with Vitamin E (density 0.95 g/cc) at a particle weight fraction of 0.333 (15% by volume of dense particles) to create a suspending vehicle. According to equation (1), the density of the suspending vehicle is 1.21 g/cc.

A small Delrin sphere (density 1.35 g/cc, 3/32″ diameter) was placed in a first container holding the suspending vehicle. After centrifuging for two minutes at 1000 rpm, the Delrin sphere sank to the bottom of the container.

A Lucite sphere (density 1.20 g/cc, 3/32″ diameter) was placed in a second container holding the suspending vehicle and centrifuged at 2000 rpm for 2 minutes. The Lucite sphere remained suspended in the suspending vehicle just beneath the surface. As a result, the ability of calcium carbonate addition to float the Lucite sphere was demonstrated.

In contrast, the Lucite sphere was placed in a third container of pure Vitamin E only and centrifuged for two minutes at 1000 rpm. In the absence of the calcium carbonate particles, the Lucite sphere sank to the bottom of the container in less than one minute.

### Example 2

### Preparation of Control Pharmaceutical Suspension

A suspending agent of density of about 1.0 g/cc is mixed with a pharmaceutically active agent comprising a density of about 1.5 g/cc to form a control pharmaceutical suspension, e.g., having no suspended density-modifying solid particles. Particles of the pharmaceutically active agent are approximately 5 to 25% of the total pharmaceutical suspension weight are mixed with a suspending agent for 15 minutes. The resulting suspension is de-aerated by placing the suspension in a vacuum oven and gently mixing with a rotating spring mixer for one hour.

### Example 3

The procedure of Example 2 is generally performed using a suspending agent comprising Vitamin E (density of about 0.95 g/cc), and particles of a pharmaceutically active agent comprising a density of about 1.4 g/cc having an average particle size of approximately 5 microns.

The pharmaceutical suspension is then tested according to the Centrifuge Test.

### Example 4

### Preparation and Testing of Pharmaceutical Suspensions

In a glass beaker using a spatula, submicron particles of a solid having an average particle size range of 100-700 nm and a density in the range of from approximately 2.0 to approximately 4.0 g/cc are mixed for about 15 minutes with a suspending agent to form a suspending vehicle. A weight ratio of a density-modifying solid to suspending agent ranges from about 2:1 to 1:2 so that the bulk suspension density of the mixture substantially matches that of a pharmaceutically active agent to be suspended, in accordance with equation (2). During mixing, the temperature can be reduced to raise the viscosity of the oil, increase shear, and enhance dispersion of the submicron particles.

An organic surfactant, such as SPAN-40, is optionally added at a concentration ranging from 0.5 to 10% of the total suspending vehicle weight to further enhance dispersion and prevent flocculation of the submicron particles.

A desired amount of spray-dried particles of a pharmaceutically active agent are added to the suspending vehicle (comprising an optional surfactant) to form a pharmaceutical suspension. The resulting mixture is stirred for an additional 15 minutes. In a preferred embodiment, the particles of the pharmaceutically active agent have a density in the range of from approximately 1.0 to 1.6 g/cc and a particle size in the range of approximately 3 to 7 microns with loadings of approximately 5 to 25% of the total suspension weight.

The resulting pharmaceutical suspension is de-aerated by placing the suspension in a vacuum oven and gently mixing with a rotating spring mixer for one hour.

### Example 5

The procedure of Example 4 is generally performed using a solid comprising zinc phosphate, a suspending agent comprising Vitamin E, and particles of a pharmaceutically active agent comprising a density of about 1.4 g/cc.

### Example 6

The procedure of Example 4 is generally performed using a solid comprising zinc silicate, a suspending agent comprising Vitamin E, and particles of a pharmaceutically active agent comprising a density of about 1.4 g/cc.

### Example 7

The procedure of Example 4 is generally performed using a solid comprising zinc chloride, a suspending agent comprising Vitamin E, and particles of a pharmaceutically active agent comprising a density of about 1.4 g/cc.

### Example 8

The procedure of Example 4 is generally performed using a solid comprising calcium phosphate, a suspending agent comprising Vitamin E, and particles of a pharmaceutically active agent comprising a density of about 1.4 g/cc.

### Example 9

The procedure of Example 4 is generally performed using a solid comprising calcium silicate, a suspending agent comprising Vitamin E, and particles of a pharmaceutically active agent comprising a density of about 1.4 g/cc.

### Example 10

### Testing of Pharmaceutical Suspensions

The pharmaceutical suspensions of Examples 5-9 are tested and analyzed according to the Centrifuge Test.

### Example 11

### Effect of Solids in Pharmaceutical Suspensions

The degree of segregation of the drug particles as determined according to Example 10 is compared with that determined according to Example 3. If the suspending vehicle is performing well, the active agent concentration profile should match the density-modifying solid concentration profile more closely than that of the drug particles in the control vehicle.

### Example 12

### Exemplary Liquid Composition

According to equation (1), a 50/50 by weight mixture of zinc chloride (density of approximately 2.9 g/cc) nanoparticles in Vitamin E (density of approximately 0.95 g/cc; viscosity of about 20 poise at room temperature) provides a suspending vehicle density of 1.43 g/cc, therefore providing suspending capacity for drug particles of that same density. The volume percent of nanoparticles in the suspending vehicle would be 25%. With a 10% by weight loading of drug particles in the overall suspension formulation, the total volume of solids would be 32% and the total solids by weight would be 55%. If the zinc chloride particles were 400 nm, they would settle at a rate of 0.7 mm/yr, neglecting Brownian Motion, which would only slow the sedimentation rate. This estimate is based on Stokes' equation (A) with a modification for hindered steeling by the Richardson-Zaki correlation. With a mismatch in density between the drug particles and the suspending vehicle of 0.05 g/cc and a drug particle size of 5 μm, the drug particles would settle at a rate of ˜2 mm/yr. The suspending vehicle viscosity would be about 70 poise and the final suspension dosage form viscosity would be about 110 poise based on equation (3) below which is a correlation for suspension viscosity ηₛᵤₛₚ as a function of solids loading given by

$${η}_{susp} = {η}_{veh} \frac{1 + 0.5 ϕ}{{( 1 - ϕ )}^{4}} , \qquad ( 3 )$$

where φ is the volume fraction of solid particulates in the suspension and ηᵥₑₕ is the viscosity of the suspending vehicle. By contrast, in the absence of the zinc chloride particles, the drug particles would settle at a rate of about 80 mm/yr.

### Example 13

### Testing of Control Liquid Composition at Lower Temperature

The pharmaceutical suspension of Example 3 is tested under a modified Centrifuge Test. Centrifugation occurs at a temperature below room temperature as low as 4° C. The lower temperature is chosen so that the viscosity of the control suspending agent according to Example 3 substantially matches that of the suspension vehicles of Examples 5-9 at room temperature. The analysis part of the Centrifuge Test is carried out.

All publications, patents and patent applications cited herein are hereby incorporated by reference in their entirety. As used in this specification and the appended claims, the singular forms “a,” “an” and “the” include plural references unless the content clearly dictates otherwise.

Other aspects of the invention will be apparent from review of the present specification and claims and all such falling within the spirit of the invention are comprehended hereby.

What is claimed:

1. A pharmaceutical suspension comprising a pharmaceutically active agent having an active agent density, ρ_{A}, and a suspending vehicle having a suspending vehicle density, ρ_{SV}; wherein the suspending vehicle density, ρ_{SV} is substantially equal to the active agent density, ρ_{A}; and wherein the suspending vehicle comprises a suspending agent having a density, ρ_{SA}, and a density-modifying solid having a solid density, ρ_{P}.

2. The pharmaceutical suspension of claim 1 which is parenterally acceptable.

3. The pharmaceutical suspension of claim 1 wherein the suspending vehicle is substantially non-aqueous.

4. The pharmaceutical suspension of claim 3 wherein a weight fraction of the density-modifying solid, X_{P}, in the suspending vehicle is determined according to formula (2):
$${X}_{P} = \frac{{ρ}_{P} * ( {ρ}_{SA} - {ρ}_{SV} )}{{ρ}_{SV} * ( {ρ}_{SA} - {ρ}_{P} )} , \qquad ( 2 ) .$$

5. The pharmaceutical suspension of claim 1 wherein the active agent density, ρ_{A}, is from about 1.0 g/cc to about 2.5 g/cc.

6. The pharmaceutical suspension of claim 5 wherein the active agent density, ρ_{A}, is from about 1.7 g/cc to about 2.5 g/cc.

7. The pharmaceutical suspension of claim 1 wherein the active agent density, ρ_{A}, and the suspending vehicle density, ρ_{SV}, differ by approximately 0.1 g/cc or less.

8. The pharmaceutical suspension of claim 7 wherein the active agent density, ρ_{A}, and the suspending vehicle density, ρ_{SV}, differ by approximately 0.05 g/cc or less.

9. The pharmaceutical suspension of claim 8 wherein the active agent density, ρ_{A}, and the suspending vehicle density, ρ_{SV}, differ by approximately 0.01 g/cc or less.

10. The pharmaceutical suspension of claim 1 that remains substantially homogenous at room temperature for at least one month without shaking.

11. The pharmaceutical suspension of claim 10 that remains substantially homogenous at room temperature for at least six months without shaking.

12. The pharmaceutical suspension of claim 11 that remains substantially homogenous at room temperature for at least one year without shaking.

13. The pharmaceutical suspension of claim 1 wherein the solid density, ρ_{P}, is at least about 2.0.

14. The pharmaceutical suspension of claim 13 wherein the solid density ρ_{P}, is at least about 2.5.

15. The pharmaceutical suspension of claim 4 wherein the suspending agent density, ρ_{SA}, is greater than or equal to 0.9 g/cc.

16. The pharmaceutical suspension of claim 15 wherein the active agent density, ρ_{A}, is from about 1.0 to about 2.5 g/cc.

17. The pharmaceutical suspension of claim 1 wherein the density-modifying solid comprises submicron particles.

18. The pharmaceutical suspension of claim 17 wherein an average particle size of the density-modifying solid is less than about 700 nm.

19. The pharmaceutical suspension of claim 1 wherein the suspending agent is Vitamin E, a vegetable oil, a lipid, or combinations thereof.

20. The pharmaceutical suspension of claim 1 wherein the density-modifying solid comprises a talc, a metal acetate, a metal ascorbate, a metal carbonate, a metal chloride, a metal oxide, a metal phosphate, a metal silicate, a metal stearate, a metal sulfate, or combinations thereof.

21. The pharmaceutical suspension of claim 20 wherein the metal is: calcium, magnesium, potassium, sodium, or zinc.

22. The pharmaceutical suspension of claim 21 wherein the density-modifying solid comprises a zinc phosphate, a zinc silicate, a calcium phosphate, a calcium silicate, a calcium carbonate, a zinc chloride, a sodium silicate, or combinations thereof.

23. A dosage form comprising:
a first wall that maintains its physical and chemical integrity during the life of the dosage form and is substantially impermeable to a pharmaceutical suspension;
a second wall that is partially permeable to an exterior fluid;
a compartment defined by the first wall and the second wall;
a pharmaceutical suspension that is positioned within the compartment and comprises a pharmaceutically active agent having an active agent density, ρ_{A}, and a suspending vehicle having a suspending vehicle density, ρ_{SV}; wherein the suspending vehicle density, ρ_{SV}, is substantially equal to the active agent density, ρ_{A} and wherein the suspending vehicle comprises a suspending agent having a suspending agent density, ρ_{SA}, and a density-modifying solid having a solid density, ρ_{P};
a pump in communication with the first wall, the second wall, and the compartment; and
an exit port in the wall in communication with the compartment.

24. The dosage form of claim 23 wherein the suspending vehicle comprises a substantially non-aqueous suspending agent.

25. The pharmaceutical suspension of claim 23 wherein a weight fraction of the density-modifying solid, X_{P}, in the suspending vehicle, is determined according to formula (2):
$${X}_{P} = \frac{{ρ}_{P} * ( {ρ}_{SA} - {ρ}_{SV} )}{{ρ}_{SV} * ( {ρ}_{SA} - {ρ}_{P} )} , \qquad ( 2 ) .$$

26. A method comprising:
identifying at least one pharmaceutically active agent having an active agent density, ρ_{A};
identifying a suspending vehicle having a suspending vehicle density, ρ_{SV}; and
determining whether the active agent density, ρ_{A}, differs from the suspending vehicle density, ρ_{SV}.

27. The method of claim 26 further comprising mixing the at least one pharmaceutically active agent with the suspending vehicle to create a pharmaceutical suspension.

28. The method of claim 26 further comprising:
identifying at least one suspending agent having a suspending agent density, ρ_{SA};
identifying at least one density-modifying solid having a solid density, ρ_{P}; and
mixing the at least one suspending agent and the at least one density-modifying solid to create the suspending vehicle.

29. The method of claim 28 further comprising mixing the at least one pharmaceutically active agent with the suspending vehicle to create a pharmaceutical suspension.

30. The method of claim 26 further comprising calculating a difference between the active agent density, ρ_{A}, and the suspending vehicle density, ρ_{SV}.

31. The method of claim 30 further comprising mixing an additional amount of the at least one suspending agent or density-modifying solid to the suspending vehicle.

32. The method of claim 28 wherein submicron particles of the at least one density-modifying solid are mixed with the at least one suspending agent.

33. The method of claim 28 wherein both the at least one suspending agent and the at least one density-modifying solid are parenterally-acceptable.

34. The method of claim 28 further comprising determining a weight fraction of the density-modifying solid, X_{P}, in the suspending vehicle, wherein the suspending vehicle density, ρ_{SV}, and the active agent density, ρ_{A} are substantially the same, according to formula (2):
$${X}_{P} = \frac{{ρ}_{P} * ( {ρ}_{SA} - {ρ}_{SV} )}{{ρ}_{SV} * ( {ρ}_{SA} - {ρ}_{P} )} , \qquad ( 2 ) .$$

35. A pharmaceutical suspension produced by the method of claim 27.

36. A method comprising administering the pharmaceutical suspension of claim 35 to a mammal.

37. The pharmaceutical suspension of claim 35 wherein the active agent density, ρ_{A}, is from about 1.0 to about 2.5 g/cc.

38. The pharmaceutical suspension of claim 35 wherein an average particle size of the at least one pharmaceutically active agent is about 10 microns or less.

39. The pharmaceutical suspension of claim 35 comprising from about 5 to 25% by weight of the pharmaceutically active agent.

40. The pharmaceutical suspension of claim 35 wherein the suspending agent is substantially non-aqueous.

41. A pharmaceutical suspension produced by the method of claim 29.

42. A method comprising administering the pharmaceutical suspension of claim 41 to a mammal.

43. The pharmaceutical suspension of claim 41 wherein the solid density, ρ_{P}, is at least about 2.0 g/cc.

44. The pharmaceutical suspension of claim 41 wherein the suspending agent density, ρ_{SA}, is greater than or equal to 0.9 g/cc.

45. The pharmaceutical suspension of claim 41 wherein an average particle size of the at least one density-modifying solid is less than about 700 nm.

46. A method comprising:
identifying a suspending vehicle having a known density, ρ_{SV};
mixing at least one pharmaceutically active agent having an active agent density, ρ_{A}, that is substantially the same as the known density, ρ_{SV}, to form a pharmaceutical suspension.

47. The method of claim 46 wherein the suspending vehicle comprises at least one suspending agent and at least one density-modifying solid.

48. The method of claim 47 wherein both the at least one suspending agent and the at least one density-modifying solid are parenterally-acceptable.

49. The method of claim 47 wherein submicron particles of the at least one density-modifying solid are mixed with the at least one suspending agent.

50. A pharmaceutical suspension produced by the method of claim 46.

51. The pharmaceutical suspension of claim 50 wherein the pharmaceutically active agent comprises a protein.

52. The pharmaceutical suspension of claim 50 wherein the pharmaceutically active agent comprises a peptide.

53. A method comprising:
identifying at least one suspending agent having a suspending agent density, ρ_{SA};
identifying at least one density-modifying solid having a solid density, ρ_{P};
mixing the suspending agent with the density-modifying solid to create a suspending vehicle having a vehicle density, ρ_{SV}; and
establishing a weight fraction of the density-modifying solid, X_{P}, in the suspending vehicle, such that the vehicle density, ρ_{SV}, is from about 1.0 g/cc to about 2.5 g/cc.

54. The method of claim 53 wherein the weight fraction is calculated according to formula (2):
$${X}_{P} = \frac{{ρ}_{P} * ( {ρ}_{SA} - {ρ}_{SV} )}{{ρ}_{SV} * ( {ρ}_{SA} - {ρ}_{P} )} , \qquad ( 2 ) .$$

55. The method of claim 53 wherein the vehicle density is from about 1.7 to about 2.5 g/cc.

56. The method of claim 53 further comprising determining whether the suspending vehicle density, ρ_{SV}, differs from an active agent density, ρ_{A}, of a pharmaceutically active agent; and mixing the pharmaceutically active agent with the suspending vehicle to create a pharmaceutical suspension.

57. The method of claim 53 further comprising identifying a biologic-compatible suspending agent.

58. A pharmaceutical suspension produced by the method of claim 56.

59. The method of claim 56 wherein both the at least one suspending agent and the at least one density-modifying solid are parenterally-acceptable.

60. The method of claim 56 wherein submicron particles of the at least one density-modifying solid are mixed with the at least one suspending agent.

61. A kit comprising a suspending vehicle produced by the method of claim 56.

62. The kit of claim 61 further comprising instructions for mixing the suspending vehicle with a pharmaceutically active agent.

63. A kit comprising a suspending agent and a density-modifying solid.

64. The kit of claim 63 further comprising instructions for preparing a mixture of the suspending agent and the density-modifying solid to form a suspending vehicle having a density that is substantially equal to an active agent density.

65. A method of making the kits of claim 61, 62, 63, or 64.
